(19) United States
(12) Patent Application Publication
(10) Pub. No.: US 2003/0146311 A1
(43) Pub. Date: Aug. 7, 2003

(54) Method for producing nanometer micro-powder of natural materials and products of the method

(76) Inventor: Chin-Hsin Yang, Taipei (TW)

Correspondence Address: Cooper & Dunham LLP, 1185 Avenue of the Americas, New York, NY, 10036, US

(21) Appl. No.: 10/330,857

(22) Filed: Dec. 26, 2002

(30) Foreign Application Priority Data
Dec. 28, 2001 (TW) 090132694

(51) Int. Cl. B02C 19/12; B02C 7/18; A61K 35/78; A61K 9/14

(52) U.S. Cl. 241/3; 424/725; 241/6

(57) ABSTRACT

A method for producing nanometer of natural materials from animals, plants or minerals containing active ingredients, the method including the steps of: obtaining raw materials from animals, plants or minerals; selecting raw materials; cleaning the raw materials; removing water from the raw materials; drying the raw materials; chopping the raw materials; crushing the raw materials; grinding the raw materials into nanometer micro-powder; and grading the nanometer micro-powder for packaging. In this method, each step is established according to different properties of the raw materials and operated at low temperature and low relative humidity to ensure the nanometer micro-powder has long preservation time and excellent efficiency to release active ingredients.

### BACKGROUND OF THE INVENTION

1. Field of the Invention

The present invention relates to a method for producing nanometer micro-powder of natural materials, and more particularly for nanometer micro-powder of animals, plants and minerals having active components that the nanometer micro-powder has long shelf life and excellent efficiency for absorbing to cure users. Products of the method are also disclosed in the present invention.

2. Description of Related Art

For many years, Western medicine has occupied the dominant position in the global pharmaceutical market. However, with increasing internationalization of daily life and exploration of Eastern culture, medicines alternative to Western chemical based types are becoming popular again, and many people make their mind open to Eastern medicine and consider more natural forms of healing as Chinese medicine emphasizing.

Because Chinese medicine uses natural materials such as herbs, animal parts, or minerals, which are believed to have moderate medical properties and has fewer side-effects than Western medicine, Chinese medicine is now used all over the world to treat with all kinds of patients. Moreover, the Western medicine uses pure compounds and creates more and more difficulty to develop new medicine (because fewer and fewer compounds remain for screening), involves long clinical trials (5-8 years), and has high development costs (billions of U.S. dollars). Therefore, Chinese medicine, such as natural material extraction from plants, animals, or minerals of compound description containing multiple active ingredients, provides another choice for medicine manufacturers and is getting popular because of the “back to nature” healing concept.

However, the conventional method for producing Chinese medicine lacks scientific technology to accurately administer the operational procedures to raw materials so that Chinese medicine deteriorates easily or does not possess appropriate amounts of active ingredients to overcome diseases. Therefore, such disadvantages of the conventional method for producing Chinese medicine need to be resolved.

### SUMMARY OF THE INVENTION

To overcome the shortcomings, the present invention provides a method for producing nanometer micro-powder of natural materials containing active components served as medicine to mitigate or obviate the aforementioned technical problems in the related art.

The main objective of the present invention is to provide a method for producing nanometer micro-powder of natural materials from plants, animals, or minerals, the method selectively produces different kinds of nanometer micro-powder in different ways in accordance with properties of the natural materials to keep active ingredients during manufacturing processes.

The other objective of the present invention is to provide a product containing the nanometer micro-powder, which has a long shelf life and excellent effects of medication to the human body.

Further benefits and advantages of the present invention will become apparent after a careful reading of the detailed description with appropriate reference to the accompanying drawing.

### BRIEF DESCRIPTION OF THE DRAWINGS

FIG. 1 is a block diagram of a method for producing nanometer micro-powder of natural materials in accordance with the present invention.

### DETAILED DESCRIPTION OF THE INVENTION

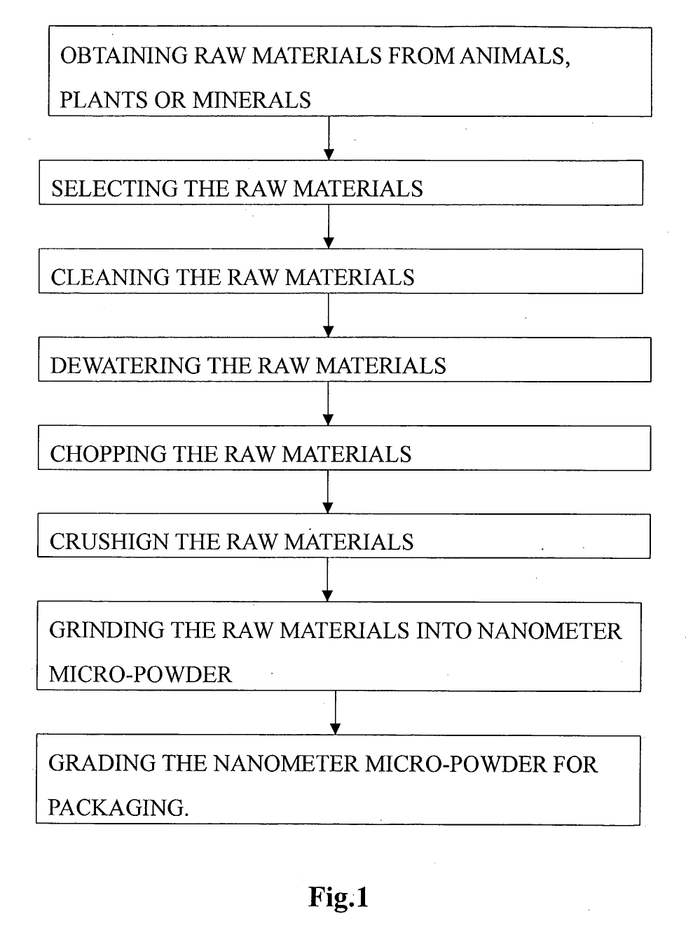

With reference to FIG. 1, a method for producing nanometer micro-powder of natural materials (plants, animals, and minerals containing active ingredients) comprises steps of: obtaining raw materials from animals, plants or minerals; selecting the raw materials; cleaning the raw materials; dewatering the raw materials; drying the raw materials; chopping the raw materials; crushing the raw materials; grinding the raw materials into nanometer micro-powder; and grading the nanometer micro-powder for packaging.

Raw materials in the present invention are natural and obtained from animals, plants, or minerals containing active ingredients.

In the selecting process, the natural raw materials from plants, animals, or minerals are obtained and selected by two stages. The first selection stage is to manually collect useful and large-size raw materials, and the second selection stage is to screen small-size raw materials by hy-sieving (using oscillating-tray applied with blowing dry-air to winnow the small-size raw materials), wherein the mesh of the hy-sieves (or the oscillating-tray) corresponds to the raw materials..

In the cleaning process, most of the selected raw materials must be washed in different washing ways according to their natural property of size or degree to release active ingredients and the washing ways comprise ultrasonic vibration cleaning, plate-dipping cleaning, and net-suspension cleaning. Because some active ingredients in the natural raw materials are easily dissolved into water or reacted with hydro-molecular to make the raw materials lose effect of medication, different raw materials have to be cleaned by a suitable washing way. For example, rhizome plants, such as ginseng, Chinese angelica root (radix angelicae sinensis), or large-flowered skullcap root (radix scutellariae), have irregular shapes and easily lose their active ingredients when washing so that the rhizome plants are adapted to be cleaned by ultrasonic vibration cleaning. Raw materials non-deteriorated in water are cleaned by plate-dipping cleaning, wherein the raw materials are placed on multiple plates and sunk into flowing water with the plates for washing. Moreover, raw materials having large-size and non-deteriorated property, such as ligneous plants or Chinese rhubarb root (Rasix et Rhizoma Rhei), are cleaned by net-suspension cleaning, wherein the raw materials are packed inside a net and dipped into flowing water for washing.

When the ultrasonic vibration cleaning operates, each batch takes 1-3 minutes to clean material. When the plate-dipping cleaning operates, each batch takes 3-15 minutes. When the net-suspension cleaning operates, each batch takes 2-15 minutes. Particularly, the ultrasonic vibrating cleaning is widely used in all kinds of materials and has the best cleaning efficiency of all washing ways. The conventional method for producing Chinese medicine usually dips the raw materials into water to clean them, however it is difficult to completely clean the raw materials and preserve active ingredients in the raw materials using that method.

Additionally, other raw materials in fine-size and easily deteriorated in water are not suitable for washing and are cleaned by vacuum air-extracting. In the vacuum air-extracting method, the raw materials are vibrated to separate dust from the raw materials into air in circumstance. The air containing dust is drawn by creating a vacuum atmosphere (or negative atmosphere) bypass to produce a moderate air flow from high atmosphere to low atmosphere to drain air with dust away.

In the process of dewatering, removal of water from the raw materials by a certain way accords to their properties of size or degree causing loss of active ingredients as described in aforementioned cleaning process. The ways for removing water are as follow: centrifuge dewatering, gravity dewatering, vibration dewatering, and squeeze dewatering.

In the drying process, the raw materials are dried by at least one of the following: hot-air circulating drying, infrared drying, and microwave drying. Raw materials possessing different properties of thermal-sensitivity are treated with a proper way. For example, raw materials in which active ingredients are not easily deteriorated by thermal exposure are treated by hot-air circulating drying at 40-90 centigrade degree, and examples of such materials include rhizome plants comprising large-flowered skullcap (Radix Scutellariae) or tuckahoe (Poria). Raw materials containing active ingredients, such as ester, gum or gelatin, are extremely thermal-sensitive and so treated with microwave drying. Moreover, infrared drying is widely used in all kinds of raw materials but has high manufacturing cost.

In the process of chopping, the raw materials are chopped with shear machine and crusher machine and then ground roughly. To raw materials which are thermal-sensitive or contain plenty of oil or sugar, such raw materials need to be dried with dry-cold air of below 45% relative humidity (RH) and below 35 centigrade degree.

Additionally, with regard to raw materials suffering thermal-sensitivity and humidity-sensitivity, the chopping operational circumstances must be controlled under negative pressure to guide cold air of below 45% relative humidity (RH) and below 35 centigrade degree.

In the crushing process, the chopped raw materials are further crushed to pieces of 40-200 mesh in a negative pressure circumstance. For thermal-sensitive raw materials, cold-air is guided into the negative pressure circumstance to reduce the temperature of the circumstance. Then, the chopped raw materials are ground with gear wheels. Raw materials containing oil, gum, gelatin, or sugar are also ground by gear wheels. Some raw materials are crushed by cutting machines or pounding machines. When crushing the chopped raw materials, the operational circumstance must be controlled below 45% relative humidity (RH) and below 35 centigrade degree.

With regard to thermal-sensitive and humidity-sensitive raw materials, the crushing operational circumstance must be controlled under negative pressure to guide cold air in the circumstance, wherein the cold air possesses relative humidity (RH) below 45% and a temperature below 35 centigrade degree.

In the grinding process, the crushed raw materials of 40-200 mesh pieces are screens by sieves to separate powder aside. The powder is guided into a fine-grinding machine and then the powder is further ground to nanometer micro-powder by the fine-grinding machine. The nanometer micro-powder removes metal impurity by an electromagnetic device and then is screened to separate the nanometer micro-powder. Remaining powder is not nanometer micro-powder and is ground by the fine-grinding machine again to achieve nanometer micro-powder.

Subsequently, the nanometer micro-powder is graded by particle diameter and packed.

According to aforementioned processes of the present method, the natural raw materials are enabled to be granulated into micro-powder less than 5-10 μm, and more particularly into nanometer micro-powder, much smaller than the 150-200 mesh (75 μm) powder produced by the conventional method. Limitations of particle size of the powder in the conventional method are mostly caused by the operational temperature is too high to make the oil, gelatin, gum, or sugar separate from the raw materials and the powder of the raw materials lose the active ingredients. Additionally, the active ingredients of the raw materials are not easily absorbed because plants enclose the active ingredients inside cell walls, which are not easily digested by the digestive system of human body. However, the nanometer micro-powder has more than 95% cell walls broken to release the active ingredient inside the raw materials so that human body easily absorbs the active ingredients.

In order to prove the efficiency of the nanometer micro-powder produced by the method in the present invention, pills of “Guifu Dihuang Wan” (Chinese prescription mainly containing cinnamon, monkshood and digitalis) are prepared for animal test.

1. Experiment Materials:

<1-1. Medicine>

1. “Guifu Dihuang Wan” A: nanometer micro-powder provided by The Medical Industrial Research Institution in Shan-Dong Province of the People Republic of China. Batch number: 961023.

2. “Guifu Dihuang Wan” B: conventional pills obtained from North Pharmacy Factory in Shan-Dong Province of the People Republic of China. Batch number: 950810.

3. “Nan-bow”: medicine for curing impotence obtained from Kang-Fu Pharmacy Factory in Ji-Lin of the People Republic of China. Batch number: 950101.

4. Oxidized cortisone: medicine obtained from Huanghe-River Pharmacy Factory in Shang-Hai of the People Republic of China. Batch number: 9411011.

The above medicines are dissolved with water in different concentrations while using.

<1-2. Animals>

Mouse: male mice of 14-16 g and 22-24 g.

Rat: Wistar male rats of 200-250 g.

2. Experiment and Results:

<2-1. Efficiency to Growth and Reproducing System of Young Male Mice>

40 young male mice were 14-16 g and divided into four groups. Four groups of mice were respectively fed with 10% “Guifu Dihuang Wan” A solution, 10% “Guifu Dihuang Wan” B solution, 5% “Nan-bow” solution, and saline, each solution was about 0.2 ml/10 g (2 g/Kg of “Guifu Dihuang Wan” A; 2 g/Kg of “Guifu Dihuang Wan” B; 1 g/1 Kg of “Nan-bow”) for two weeks. After 24 hours, blood was drawn from the mice to test the quantity of serum testosterone in the blood. After two weeks, the mice were dissected to obtain testis, prostate, levator ani, and musculus bulbocavernosus for weighing. Results of the experiment were listed in Table 1.

TABLE 1

| | | | | | | Levator ani and |
|---|---|---|---|---|---|---|
| | | | Testos- | Prostate and | | musculus |
| | | Dosage | Terone | spermary | | bulbocavernosus |
| Group | Number | (g/kg) | (μg/DL) | (g/10 g) | Testis (g/10 g) | (g/10 g) |
| Control (saline) | 10 | | 10.75 ± 1.39 | 21.74 ± 10.00 | 54.64 ± 10.17 | 20.29 ± 5.70 |
| “Nan-bow” | 10 | 1.0 | 14.85 ± 8.74 | 19.54 ± 9.30 | 61.44 ± 12.73 | 17.44 ± 4.09 |
| “Guifu Dihuang | 10 | 2.0 | 11.13 ± 2.98 | 17.96 ± 5.10 | 59.53 ± 9.28 | 19.78 ± 3.90 |
| Wan” B | | | | | | |
| Guifu Dihuang | 10 | 2.0 | 14.06 ± 2.21² | 25.61 ± 14.87 | 60.02 ± 8.00 | 27.74 ± 6.31¹ |
| Wan A | | | | | | |

In comparison with Control: ¹P<0.05, ²P<0.001

Table 1 shows that the group of “Guifu Dihuang Wan” A increases the testosterone quantity, weights of levator ani and musculus bulbocavernosus of the young male mice in comparison with the control of saline. The group of “Guifu Dihuang Wan” B has no obvious change in comparison with the control of saline. Meanwhile, “Guifu Dihuang Wan” A has greater efficiency to growth than “Guifu Dihuang Wan” B (not shown in table 1).

<2-2. Efficiency to Impotent Rats>

50 male rats were divided into five groups and all rats were castrated except a model group. Four groups of castrated rats were respectively fed with 10% “Guifu Dihuang Wan” A solution, 10% “Guifu Dihuang Wan” B solution, 5% “Nan-bow” solution, and 1.5 g/Kg saline, each solution was about 0.2 ml/10 g dosage (1.5 g/Kg of “Guifu Dihuang Wan” A; 1.5 g/Kg of “Guif ti Dihuang Wan” B; 0.75 g/Kg of “Nan-bow”) for two weeks. After 24 hours, blood was drawn from the rats to test the quantity of serum corticosterone in the blood. After two weeks, the rats were dissected to obtain gonophore for weighing. Results of this experiment were listed in Table 2.

TABLE 2

| | | | | | Levator ani and |
|---|---|---|---|---|---|
| | | Dosage | Corticosterone | | musculus |
| Groups | number | (g/kg) | (μg/DL) | Testis (g/10 g) | bulbocavernosus (g/10 g) |
| Control | 8 | — | 0.792 ± 0.0573 | 2.840 ± 0.420 | 2.448 ± 0.928 |
| Model | 9 | — | 0.171 ± 0.038 | 0.192 ± 0.080 | 0.685 ± 0.254 |
| “Nan-bow” | 7 | 0.75 | 0.237 ± 0.0393 | 0.207 ± 0.101 | 0.731 ± 0.276 |
| “Guifu Dihuang Wan” | | | | | |
| B | 8 | 1.5 | 0.473 ± 0.0683 | 0.455 ± 0.286 | 1.031 ± 0.305 |
| “Guifu Dihuang Wan” | | | | | |
| A | 8 | 1.5 | 0.490 ± 0.0643 | 0.306 ± 0.0632 | 0.938 ± 0.064 |

In comparison with Model P<0.05:P<0.01:P<0.001

<2-3. Efficiency to Mice>

50 male mice were divided into five groups, wherein one group was control and one group was model, and both said groups were treated with saline. The other three groups were respectively fed with 2 g/2 Kg “Guifu Dihuang Wan” A solution, 2 g/2 Kg “Guifu Dihuang Wan” B solution, 1 g/1 Kg “Nan-bow” solution once a day for two weeks. The mice were injected with 25 mg/Kg oxidized cortisone on the 11^{th} day for 3 more days. After two weeks, the temperatures of the mice were recorded, times of activity within 5 minutes, swimming times, weight of immune organs and procreating organs. The results of this experiment were shown in Tables 3-5.

TABLE 3

| | | Dosage | Weight | Times of activity | Swimming time | Temperature |
|---|---|---|---|---|---|---|
| Groups | Number | (g/kg) | (g) | (within 5 min) | (min) | (° C) |
| Control | 10 | — | 32.96 ± 5.81 | 8.11 ± 69.04 | 39.10 ± 18.95 | 37.55 ± 0.92² |
| Model | 10 | — | 28.94 ± 3.64 | 8.22 ± 5.38 | 15.8 ± 4.76 | 33.94 ± 0.06 |
| “Nan-bow” | 10 | 1.0 | 34.00 ± 2.42 | 72.11 ± 23.09 | 38.78 ± 60.17 | 37.70 ± 0.62² |
| “Guifu Dihuang | 10 | 2.0 | 33.89 ± 3.19 | 82.89 ± 34.33 | 41.22 ± 27.17 | 37.98 ± 0.38² |
| Wan” B | | | | | | |
| “Guifu Dihuang | 10 | 3.0 | 33.78 ± 4.69 | 77.56 ± 24.08 | 75.67 ± 43.29 | 37.52 ± 0.59² |
| Wan” | | | | | | |
| A | | | | | | |

In comparison with Model:
P < 0.05;
²P < 0.01;
P < 0.001

TABLE 4

| | | | | | Levator ani and |
|---|---|---|---|---|---|
| | | | | Prostate and | musculus |
| | | Dosage | Testis | spermary | bulbocavernosus |
| Groups | number | (g/kg) | (g/10 g) | (g/10 g) | (g/10 g) |
| Control | 10 | — | 57.25 ± 9.03 | 51.83 ± 14.88 | 38.72 ± 15.45 |
| Model | 10 | — | 59.93 ± 11.79 | 41.77 ± 26.60 | 26.33 ± 5.09 |
| “Nan-bow” | 10 | 1.0 | 57.40 ± 8.07 | 63.78 ± 18.20 | 28.29 ± 4.26 |
| “Guifu Dihuang Wan” | 10 | 2.0 | 66.59 ± 15.96 | 54.72 ± 7.45 | 28.87 ± 4.39 |
| B | | | | | |
| “Guifu Dihuang Wan” | 10 | 2.0 | 63.45 ± 8.66 | 64.48 ± 9.75 | 31.88 ± 4.42 |
| A | | | | | |

In comparison: P < 0.05.

TABLE 5

| | | Dosage | Thymus gland | Spleen |
|---|---|---|---|---|
| Groups | Number | (g/kg) | (mg/10 g) | (mg/10 g) |
| Control | 10 | — | 22.66 ± 7.03 | 55.58 ± 19.76 |
| Model | 10 | — | 6.55 ± 1.77 | 34.54 ± 11.73 |
| “Nan-bow” | 10 | 1.0 | 20.61 ± 0.88 | 44.28 ± 4.36 |
| “Guifu Dihuang | 10 | 2.0 | 17.95 ± 0.89 | 45.26 ± 8.43 |
| Wan” B | | | | |
| “Guifu Dihuang | 10 | 2.0 | | |
| Wan” A | | | | |

According to the above experiment, the “Guifu Dihuang Wan” A containing nanometer micro-powder in the present invention has excellent efficiency in comparison to other medicine made by the conventional method. For example, “Guifu Dihuang Wan” A increases the quantity of serum testosterone and weight of procreating organ much more than “Guifu Dihuang Wan” B. That is to say, the nanometer micro-powder releases active ingredients efficiently.

Nanometer micro-powder made by the method in accordance with the present invention is applied to various industries such as medicine composition, cosmetics, or health comestibles, and more particular as Chinese medication. The Chinese medication containing nanometer micro-powder or medicament of nanometer micro-powder is selectively taken by inhaling the nanometer micro-powder, eating the nanometer micro-powder or drinking after decocting. With regard to health comestibles, the nanometer micro-powder is selectively added in food in combination with edible substance of solid, liquid, or gel. Additionally, the health comestibles further contain condiments and pigments and are eaten directly or after dissolving in water.

In the method of the present invention, each process is established according to the properties of different raw materials and the operational conditions are controlled to ensure the active ingredients are kept in the raw material when producing the nanometer micro-powder.

The nanometer micro-powder contains only 7-9% water which prevents deterioration of the nanometer micro-powder. Additionally, the nanometer micro-powder is smaller than eggs of insect pests common in this field so that those eggs can be screened and excluded from the nanometer micro-powder during manufacturing. Therefore, the nanometer micro-powder is not contaminated by insect eggs and does not easily decay even when no antiseptic or preservative are contained in the health comestibles.

Moreover, the diameter of the nanometer micro-powder grain is also smaller than diameter of human capillary so that the nanometer micro-powder can permeate into skin and be absorbed directly such that it is suitable as an inhalation medication, dressing medication or cosmetics. Take a facial dressing for example, the dressing materials can be 100% nanometer micro-powder and do not need other inactive excipient or plasticizer such as starch, gum, or kaolin. The dressing materials can be partially made of nanometer micro-powder of multiple raw materials. Take the health comestibles or a medicament for example, the nanometer micro-powder can be added into carrier in forms of liquid, solid, or glue for application.

With regard to decocting, the raw materials usually take 180-240 minutes to achieve the effective medicine soup in a conventional decocting method. However, raw materials in forms of nanometer micro-powder in the present invention easily achieve an effective medicine soup after boiling within 25 minutes (about 15 minutes) because the active ingredient are easily released from the broken cell walls.

Various modifications and variations of the present invention will be recognized by those persons skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

What is claimed is:

1. A method for producing nanometer micro-powder of natural materials from animals, plants or minerals containing active ingredients, the method comprising the following steps:
obtaining raw materials from animals, plants or minerals;
selecting the raw materials;
cleaning the raw materials;
dewatering the raw materials;
drying the raw materials;
chopping the raw materials, wherein an operational temperature is below 35 centigrade degree and a relative humidity is below 45%;
crushing the raw materials, wherein the raw materials are crushed to pieces of 40-200 mesh and an operational temperature is below 35 centigrade degree and a relative humidity is below 45%; and
grinding the raw materials of pieces into nanometer micro-powder.

2. The method as claimed in claim 1, wherein in the cleaning step, the raw materials are washed using ultrasonic vibration cleaning, plate-dipping cleaning or net-suspension cleaning, or a combination of at least two thereof.

3. The method as claimed in claim 2, wherein the raw materials are washed using ultrasonic vibration cleaning for 1 to 3 minutes for a batch.

4. The method as claimed in claim 2, wherein the raw materials are washed using ultrasonic vibration cleaning for 3 to 15 minutes for a batch.

5. The method as claimed in claim 2, wherein the raw materials are washed using ultrasonic vibration cleaning for 2 to 15 minutes for a batch.

6. The method as claimed in claim 1, wherein in the dewatering step, the raw materials are dewatered using centrifugal dewatering, gravity dewatering, vibrating dewatering or squeeze dewatering, or a combination of at least two thereof.

7. The method as claimed in claim 1, wherein in the drying step, the raw materials are dried using hot-air circulating drying, infrared drying or microwave drying, or a combination of at least two thereof.

8. The method as claimed in claim 7, wherein the raw materials are dried using hot-air circulating drying at a temperature of from 40 to 90 centigrade degree.

9. The method as claimed in claim 1, wherein the chopping step is carried out at a relative humidity below 45%.

10. The method as claimed in claim 1, wherein the chopping step is carried out at a temperature below 35 centigrade degree.

11. The method as claimed in claim 1, wherein the crushing step is carried out at a relative humidity below 45%.

12. The method as claimed in claim 1, wherein the crushing step is carried out at a temperature below 35 centigrade degree.

13. A method for cleaning natural raw materials, wherein the raw materials are washed using ultrasonicsonic vibration cleaning.

14. The method for cleaning as claimed in claim 13, wherein the raw materials are washed usingultrasonic vibration cleaning for 1 to 3 minutes for a batch.

15. A method for chopping thermal-sensitive and humidity-sensitive raw materials, in which an operational temperature is below 35 centigrade degree and a relative humidity is below 45%.

16. The method for chopping as claimed in claim 15, wherein the relative humidity is below 45%.

17. The method for chopping as claimed in claim 15, wherein the operational temperature is below 35 centigrade degree.

18. The method for chopping as claimed in claim 17, wherein the operational temperature is maintained by guiding cold air in a negative pressure circumstance.

19. A method for crushing thermal-sensitive and humidity-sensitive raw materials, wherein the raw materials are crushed to pieces of 40-200 mesh at a temperature of below 35 centigrade degree; and at a relative humidity of below 45%.

20. The method as claimed in claim 19, wherein the raw materials are crushed at a relative humidity of below 45%.

21. The method for crushing as claimed in claim 19, wherein the operational temperature is below 35 centigrade degree.

22. The method for crushing as claimed in claim 21, wherein the operational temperature is maintained by guiding cold air in a negative pressure circumstance.

23. A pharmaceutical composition comprising a therapeutically effective amount of nanometer micro-powder, wherein the nanometer micro-powder is made by following steps:
obtaining raw materials from animals, plants or minerals;
selecting raw materials;
cleaning the raw materials;
dewatering the raw materials;
drying the raw materials;
chopping the raw materials, wherein an operational temperature is below 35 centigrade degree and a relative humidity is below 45%;
crushing the raw materials, wherein the raw materials are crushed to pieces of 40-200 mesh and an operational temperature is below 35 centigrade degree and a relative humidity is below 45%; and
grinding the raw materials of pieces into nanometer micro-powder.

24. The medicine composition as claimed in claim 23, wherein in the cleaning step, the raw materials are washed using ultrasonic vibration cleaning, plate-dipping cleaning, or net-suspension cleaning or a combination of at least two thereof.

25. The medicine composition as claimed in claim 24, wherein the raw materials are washed using ultrasonic vibration cleaning for 1 to 3 minutes for a batch.

26. The medicine composition as claimed in claim 24, wherein the raw materials are washed using plate-dipping cleaning for 3 to 15 minutes for a batch.

27. The medicine composition as claimed in claim 24, wherein the raw materials are washed using net-suspension cleaning for 2 to 15 minutes for a batch.

28. The medicine composition as claimed in claim 23, wherein in the dewatering step, the raw materials are dewatered using centrifugal dewatering, gravity dewatering, vibrating dewatering, or squeeze dewatering, or a combination of at least two thereof.

29. The medicine composition as claimed in claim 23, wherein in the drying step, the raw materials are dried using hot-air circulating drying, infrared drying, or microwave drying, or a combination of at least two thereof.

30. The medicine composition as claimed in claim 29, wherein the hot-air circulating drying has a temperature range of 40-90 centigrade degree.

31. The medicine composition comprising nanometer micro-powder as claimed in claim 23, wherein the medicine composition is applied to skin dressing.

32. The medicine composition comprising nanometer micro-powder as claimed in claim 23, wherein the medicine composition is applied to inhalation therapy.

33. A health comestible comprising nanometer micro-powder and carrier selected from edible substance in forms of liquid, solid, or glue, wherein the nanometer micro-powder is made by the following steps:
obtaining raw materials from animals, plants or minerals;
selecting raw materials;
cleaning the raw materials;
dewatering the raw materials;
drying the raw materials;
chopping the raw materials, wherein an operational temperature is below 35 centigrade degree and a relative humidity is below 45%;
crushing the raw materials, wherein the raw materials are crushed to pieces of 40-200 mesh and an operational temperature is below 35 centigrade degree and a relative humidity is below 45%; and
grinding the pieces of the raw materials into nanometer micro-powder.

34. The health comestible as claimed in claim 33, wherein in the cleaning step, the raw materials are washed using ultrasonic vibration cleaning, plate-dipping cleaning, or net-suspension cleaning, or a combination of at least two thereof.

35. The health comestible as claimed in claim 34, wherein the raw materials are washed using ultrasonic vibration cleaning for 1 to 3 minutes for a batch.

36. The health comestible as claimed in claim 34, wherein the raw materials are washed using plate-dipping cleaning for 3 to 15 minutes for a batch.

37. The health comestible claimed in claim 34, wherein the raw materials are washed using net-suspension cleaning for 2 to 15 minutes for a batch.

38. The health comestible as claimed in claim 33, wherein in the dewatering step, the raw materials are dewatered using centrifugal dewatering, gravity dewatering, vibrating dewatering, or squeeze dewatering, or a combination of at least two thereof.

39. The health comestible as claimed in claim 33, wherein in the drying step, the raw materials are dried using hot-air circulating drying, infrared drying, or microwave drying, or a combination of at least two thereof.

40. The health comestible as claimed in claim 39, wherein the hot-air circulating drying has a temperature range of 40-90 centigrade degree.

41. The health comestible as claimed in claim 33, wherein the health comestible further contains condiments and pigments.
